(19) United States
(12) Patent Application Publication
(10) Pub. No.: US 2025/0261888 A1
(43) Pub. Date: Aug. 21, 2025

(54) DEVICE FOR MONITORING THE PSYCHOEMOTIONAL STATE OF A USER

(71) Applicant: Autism Care, LLC, New York, NY (US)

(72) Inventors: Vitalii VLASOV, Novosibirsk (RU); Anna KOTLOVA, Karaga (RU)

(21) Appl. No.: 18/582,131

(22) Filed: Feb. 20, 2024

(51) Int. Cl. A61B 5/16; A61B 5/00; A61B 5/024; A61B 5/0533; G16H 10/20; G16H 20/70; G16H 50/20

(52) U.S. Cl. CPC A61B 5/165; A61B 5/02405; A61B 5/02416; A61B 5/0533; A61B 5/681; A61B 5/725; A61B 5/7267; A61B 5/743; G16H 10/20; G16H 20/70; G16H 50/20

(57) ABSTRACT

A device for monitoring a psycho-emotional state of a user relates to electronic devices for collecting and processing user data for the purpose of monitoring the psycho-emotional state. The present invention comprises a photoplethysmogram sensor, a microcontroller, an accelerometer, an electrodermal activity (EDA) sensor, and an analysis unit configured to generate data for assessing the psycho-emotional state of the user based on signals from the said sensors, wherein the analysis unit is configured to perform preliminary analysis of data from the accelerometer and photoplethysmogram sensor to determine periods and levels of the user's physical activity during the said generation of the data for assessing the psycho-emotional state of the user. The objective of the present invention is to provide a wearable compact device configured to record the physiological parameters of the user with high accuracy and transmit these data wirelessly. This objective is solved according to the present invention by achieving a technical result consisting in expanding the device possibilities for monitoring the user's state indicators through using several sensors of the user's physiological indicators simultaneously, the data of which sensors are analyzed and corrected, allowing to assess the user's state even during periods of activity, and also by using compact and not power-consuming sensors and other parts of the device, which allows to make the device wearable.

### FIELD OF THE INVENTION

The claimed invention relates to electronic devices providing collection and processing of user data for the purpose of psycho-emotional state monitoring.

### STATE OF THE ART

Obtaining information about the person's state, in particular on the basis of objective physiological indicators correlating with the psycho-emotional state of a person, is of concern in many spheres of life and industry including medicine, social services, security systems and many others. At the same time, one of the most critical things for the solution of monitoring tasks is the detection of stress state.

Some prior art devices for monitoring a psycho-emotional state of a user and their components are based primarily on pulse oxymetry sensors, which does not provide a complete picture of emotional arousal and stress level, since pulse oxymetry indicators are sensitive not only to changes in emotional background, but also to physical activity.

To address this problem, such devices can be augmented with other data such as skin electrical activity data, data about person's movements, heartbeat data, etc.

Electrodermal activity (EDA) combines a number of indicators such as skin galvanic response (SGR), skin potential level SPL, skin potential response SPR, spontaneous skin potential response SSPR, skin resistance level SRL or skin conductance level SCL, skin resistance response SRR or skin conductance response SCR, spontaneous skin resistance response SSRR or spontaneous skin conductance response SSCR. There are a number of methods and devices for obtaining data on the level and changes in skin potential, resistance, and conductance. Most of these devices comprises electrodes that are in contact or at a short distance from the body to create a conductance or potential difference between them, which provides the ability to generate signals based on the above-mentioned indicators. Data on the electrical activity of the skin can be obtained using sensors of skin-galvanic response (SGR), which have a number of disadvantages such as polarization of contacts, skin habituation to the constant current flow, the need for placement on certain parts of the body, and possible allergic reactions to the chemical composition of the electrodes. Also, SGR does not cover the entire electrodermal activity (EDA) data set.

An invention is known (US 20220304603 A1; publ. Sep, 29, 2016; IPC: A61B 5/16; A61B 5/00; A61B 5/0205; A61B 5/024; A61B 5/0533; A61B 5/08; A61B 5/33; G16H 20/70; G16H 50/20) disclosing: one or more physiological sensors configured to interact with the body of a user, and a transmitter configured to receive and transmit data obtained by the one or more physiological sensors, one or more processors coupled to the emotional state monitoring device with an option of data exchange and configured to receive data from the transmitter, wherein the one or more processors have a memory for storing instructions, which instructions, when executed, cause the one or more processors: to receive, via the one or more physiological sensors, data corresponding to one or more physiological parameters of the user; to detect, via at least one of the one or more physiological sensors, a change in at least one of the one or more physiological parameters; to request, via the emotional state monitoring device, an emotional state identifier corresponding to the change; and to provide, via the emotional state monitoring device, a suggested response based on the emotional state identifier.

Disadvantages of the said invention include the fact that it does not provide specific embodiments of the said sensors or combinations thereof in a form suitable for producing compact wearable device and ensuring compliance with a number of requirements related to user-friendliness, absence of significant instrumental errors, ability to operate from a compact power source, and possibility for determining the psycho-emotional stress of the user.

Electrodermal activity (EDA) sensors are also known from the prior art, for example, an application (RU 2022110240 A; publ. Oct. 16, 2023; IPC: A61B 5/16; A61B 5/053) providing a sensor discloses a single-electrode sensor of electrical activity of the surface layer of the user's body comprising: one electrode comprising a set of conductors and configured to be placed on the user's body or at a distance therefrom providing capacitive and/or inductive “electrode-body” coupling; an analysis unit configured to determine one or more indicators of electrical activity of the surface layer of the body on the basis of at least one of the following signals: leakage current of the “electrode-body” capacitor, capacitance of the “electrode-body” capacitor, inductance of the “electrode-body” system, a signal generated by an external electromagnetic field and passing through the said capacitor, and modulation of the phase, frequency and/or amplitude of the said signal. The proposed sensor is based on the reading a secluded conductor potential, which reading is performed by adjusting a pulse signal generator.

Disadvantages of the said invention include the fact that such a sensor is low-responsive, i. e., it demonstrates a relatively small change in frequency with a relatively small change in capacitance, reducing the accuracy of measurements.

An invention is also known (US 2016262690 A1; publ. Sep. 15, 2016; IPC: A61B 5/00; A61B 5/0205) disclosing a sleep quality management device comprising a sensor module and a processing unit. The sensor module is configured to provide a heart rate signal and a skin conductance signal. The processing unit is coupled to the sensor module. The processing unit is configured to determine a sleep stage and a stress level according to the heart rate signal and the skin conductance signal, so as to identify the occurrence of a stress dream. The occurrence of stress dreaming is identified when the sleep stage corresponds to a rapid eye movement (REM) stage and the stress level corresponds to a stressed state.

It is also a disadvantage of the said invention that it does not describe possibilities of producing the device in a wearable form. Also, the operation of the device is described only for a sleeping user, while its ability to operate when the user is active is not described.

Disadvantages of all above-mentioned inventions are insufficient measurement accuracy or the inability to make the device in a compact form, in order to produce a wearable device.

### SUMMARY OF THE INVENTION

The objective of the present invention is to provide a wearable compact device configured to record physiological indicators of a user with high accuracy and transmit these data wirelessly. Specific physiological indicators read by the device are at least EDA, ECG or plethysmogram and body position changes, based on which a psycho-emotional state of the user can be assessed.

This problem is solved by achieving in the present invention a technical result consisting in expanding device capabilities for monitoring the user's condition indicators. Wider range of possibilities is provided by using several sensors of user's physiological indicators simultaneously, with the data from these sensors being analyzed and corrected, which makes it possible to assess the state of the user even during periods of activity, and also by using compact and not power-consuming sensors and other parts of the device, which allows to make the device wearable.

According to one aspect of the invention, a wearable device is provided for monitoring user's state, the device comprises a microcontroller, a photoplethysmogram sensor, an accelerometer, a skin electrical activity sensor, and an analysis unit configured to generate data about assessing the condition of the user based on signals from the said sensors, wherein the analysis unit is configured to perform preliminary analysis of data from the accelerometer and photoplethysmogram sensor to determine periods and levels of user's physical activity when generating data on the assessment of the psycho-emotional state of the user, which allows to increase the accuracy not only for determining the periods of physical activity and heart rate variability data, but also for the assessment data generated, which directly contributes to the achievement of the technical result.

The device may additionally comprise a muscle activity sensor, a light sensor, a temperature sensor, a humidity sensor, a sound recording device, wherein the analysis unit is configured to take into account the data from the said sensors to adjust the results of the psycho-emotional state assessment.

These and other aspects of the invention will be explained and understood with reference to the embodiments of the invention described further in the present application.

### DESCRIPTION OF THE DRAWINGS

The subject matter of the claims of the present application is described, claim by claim, and is clearly stated in the claims. The above-mentioned objectives, features and advantages of the invention are apparent from the following detailed description, in conjunction with the accompanying drawings showing:

FIG. 1 shows a schematic diagram of a device for monitoring a psycho-emotional state of a user, according to the present invention.

FIG. 2 shows a schematic diagram of the device for monitoring a psycho-emotional state of a user using a temperature sensor and a muscle activity sensor, according to the present invention.

FIG. 3 shows an embodiment of EDA sensor with two electrodes based on reading the time of charge generated by the electrodes, according to the present invention.

FIG. 4 shows an embodiment of EDA sensor with two electrodes based on reading the average discharge current of the electrodes, according to the present invention.

FIG. 5 shows an embodiment of interdigital type electrode.

FIG. 6 shows a functional diagram of a device with wireless data transmission, according to the present invention.

FIG. 7 shows an example of calculated acceleration data plotted as a function of time, obtained over a 30 minute period, based on the accelerometer data.

FIG. 8 shows an example of device data obtained over a 30 minute period and plotted as a function of time, which device comprises an EDA sensor, a photoplethysmogram sensor, and an accelerometer.

These drawings are explained by the following designations: 1 is Microcontroller (MCU); 2 is Photoplethysmogram Sensor (PPG Sensor); 3 is Accelerometer (MT); 4 is EDA Sensor; 5 is Battery; 6 is Memory Device; 7 is Charging Circuit; 8 is Temperature Sensor; 9 is Muscle Activity Sensor; 10 is Strapping; 11 is Electrode; 12 is Amplifier; 13 is Filter; 14 is Mobile Application; 15 is Analysis Unit; 16 is Stress Level.

### DETAILED DESCRIPTION OF THE INVENTION

In the following detailed description of the embodiments of the invention, numerous implementation details are provided to ensure a clear understanding of the present invention. However, it will be obvious to those skilled in the art how the present invention can be realized with or without these implementation details. In other instances, well-known methods, procedures and components are not described in detail so as not to impede an undue explanation of the features of the present invention.

Furthermore, it is clear from the foregoing disclosure that the invention is not limited to the described embodiments. Numerous possible modifications, changes, variations and substitutions will be obvious to those skilled in the art without departure from the scope and spirit of the present invention.

Hereinafter, “sensor” refers to a collection of sensors (devices for taking readings from a person's body) and components that convert data received from the sensors.

Ensuring adequate response to stress is the basis for maintaining homeostasis in the human body. The main regulatory systems involved in the formation of the stress response are the hypothalamic-pituitary-adrenal axis (HPA), the autonomic nervous system (ANS), and the central nervous system (CNS). Heart rate (HR) and rhythm are largely under the control of the ANS, although the heart muscle also has the ability to independently control its contractions (automaticity). HR is controlled by the combined balanced influence of the sympathetic (SNS) and parasympathetic (PNS) nervous system. The parasympathetic influence on HR is mediated by the release of acetylcholine from the vagus nerve. Muscarinic acetylcholine receptors respond to this release by increasing the permeability of the cell membrane to the K+ ion. The sympathetic effect on HR is mediated by the release of adrenaline and noradrenaline. β-adrenergic receptors are activated by the release of these hormones, resulting in cAMP-mediated phosphorylation of membrane proteins. An increase in SNS activity or a decrease in PNS activity results in an increase in HR, whereas a decrease in SNS activity or an increase in PNS activity causes a decrease in HR. SNS mainly exerts effect on the ventricular muscles of the heart and increases their contractility. In addition, SNS increases the excitation rate, conduction velocity, and excitability of the sinoatrial (SA) node. When SNS stimulation is maximized, the magnitude of HR and contractility can triple and double, respectively. The PNS primarily acts on the sinoatrial and atrioventricular (AV) nodes to reduce HR. Vagal and sympathetic activity are in constant interaction. Autonomic activity modulates HR, so the degree of HR variability provides information about the state and functioning of neural regulation and about the heart's ability to respond to these stimuli. Because the heart is not a metronome and its beats are irregular, heart rate variability (HRV) is normal and expected. In addition, HRV indicates the heart's ability to respond to and compensate for multiple body events (e. g., respiration, exercise, mental stress, hemodynamic and metabolic changes, sleep, and orthostatism).

Therefore, heart rate variability (HRV) can be used as a valuable tool to measure sympathetic and parasympathetic activity of the ANS, which in turn is directly involved in shaping the stress response.

Within the scope of the proposed device, HRV data can be obtained from ECG or plethysmogram, in particular by calculating the intervals between P-P peaks.

In this case, according to the proposed invention, it is also meant to take into account the indicators of the accelerometer located in the proposed device, which makes it possible not only to increase the accuracy of heartbeat detection, but also to determine periods of physical activity and, in turn, to make a more accurate determination of the emotional state due to the ability to take into account these data. In addition, on the basis of the accelerometer readings, it is possible to determine changes in the character of movements, in particular, to recognize changes which, in conjunction with the readings of the electrodermal activity sensor and ECG data, with a high probability indicate repetitive movements characteristic of a feeling of anxiety, or a decrease in activity associated with fatigue and apathy, therefore directly contributing to the achievement of the specified technical result.

For monitoring, the device may be in the form of a wide variety of wearable accessories or clothing elements, such as a bracelet, a ring, a patch, a device integrated into clothing or footwear, and other similar elements.

According to a preferred embodiment, the proposed device includes a wearable device comprising an electronics unit and a sensor unit. The electronics unit comprises, but not limited to, a processor, a rechargeable power supply, antennas and their strapping, a memory device, a wireless communication device, and other components.

The sensor unit comprises at least one EDA sensor, a photoplethysmogram sensor, an accelerometer, and, according to some embodiments, at least some of the components such as a temperature sensor, a muscle activity sensor (electromyograph), sound recorders, light sensors: a light sensor of UVA/UVB, RGB-IR and other spectra, an ambient temperature sensor, a humidity sensor, and so on.

According to one embodiment of a minimal variant of the proposed wearable device for monitoring a psycho-emotional state of a user, the device comprises a photoplethysmogram sensor, an accelerometer and an EDA sensor.

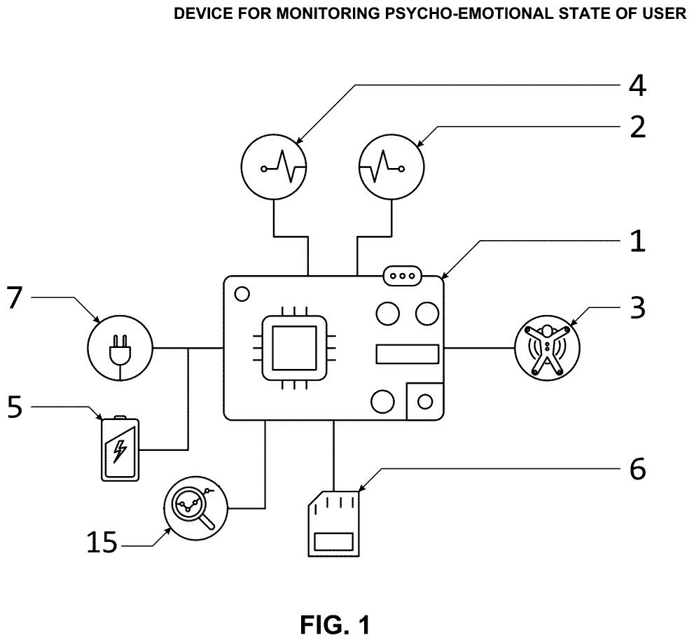

FIG. 1 shows a structural diagram of one embodiment of the device comprising the minimum equipment. According to this embodiment, the device comprises a photoplethysmogram sensor, an accelerometer, an EDA sensor, a microcontroller and an analysis unit. FIG. 1 also shows such elements as a battery, a memory device and a charging circuit, and it will be clear to those skilled in the art that these elements are not the subject of the present invention and can be implemented in various ways without significantly affecting the solution of the above-mentioned problems. During some time interval predetermined in the device, the microcontroller reads data from the photoplethysmogram sensor, accelerometer and EDA sensor, after which these data are sent to the analysis unit, where they are preprocessed, which preprocessing depends on the user's movements. According to the most general embodiment, the more active user's movements are recorded, the stronger processing is required to obtain data about the psycho-emotional state of the user based on the said data from the device components. If the user is actively moving, the readings from the sensors become more noisy, in which case more resource-intensive so-called strong processing methods are required, such as the application of the Hampel filter, but other strong processing methods are also acceptable, which will be obvious to those skilled in the art. Further, a signal processing in time is performed and indicators of the psycho-emotional state of the user are determined, for example, the level of stress, and such implementation directly contributes to the achievement of the technical result. It should be noted that structurally the analysis unit can be located in the device housing as well as can be distributed, i. e. have some remote computing power, with wireless communication between them. According to some embodiments, the invention may also be used in conjunction with a third-party mobile application, that is, data from the analysis unit may also be received by some remote device, on which such an application is installed, being configured to receive data from the analysis unit.

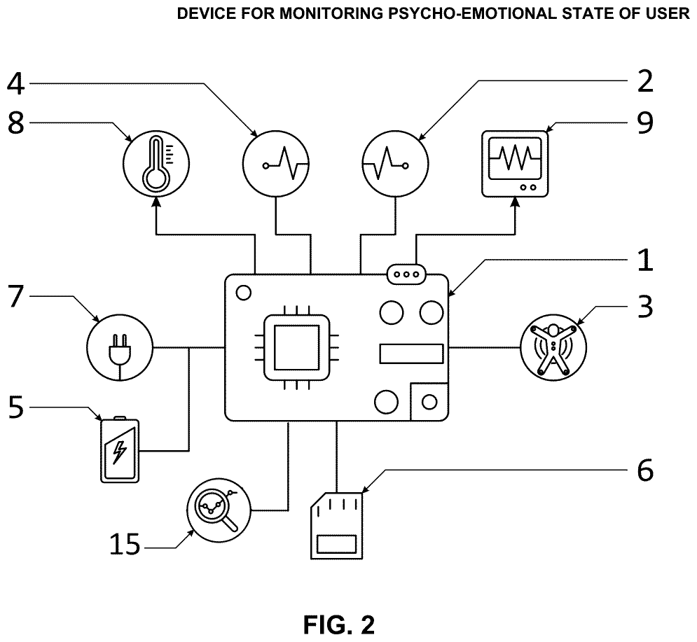

FIG. 2 shows a schematic diagram of an embodiment additionally comprising a temperature sensor and a muscle activity sensor. Although this embodiment may have higher power consumption and larger size, compared to the minimum configuration described above, due to the presence of additional sensors, on the other hand, additional sensors allow better filtering of output data for the avoidance of any influence of ambient temperature and muscle activity, which influence makes it difficult to obtain objective data indicating the psycho-emotional state of the user and plays a role of noise source when reading data. When operating the device, according to this embodiment, data from the sensor of temperature, environment and sensor of muscle activity (myogram) are taken into account by the analysis unit at least at the step of preprocessing, as a result of which it is possible to compensate for distortions and noise introduced by temperature and movements, which also improves the technical result.

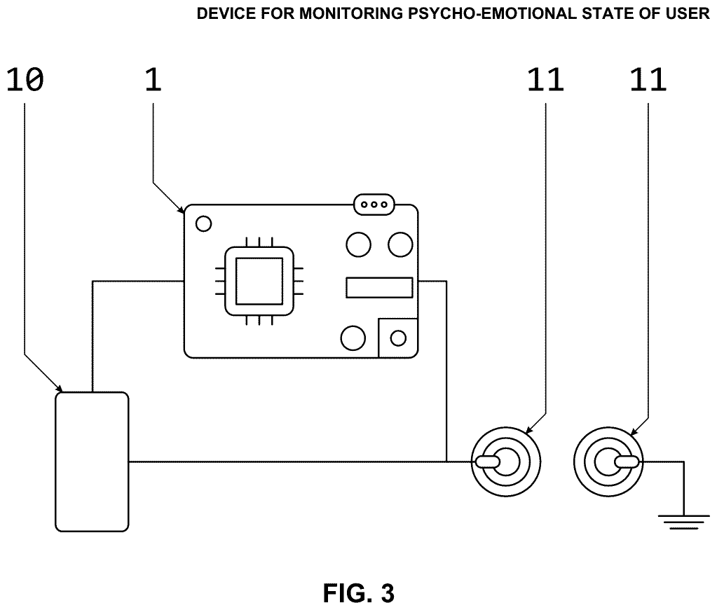

One of the embodiments of the EDA sensor shown in FIG. 3 is based on reading the charge time of the capacitor formed by the electrodes by means of a charge current limiting strapping, for this embodiment a two-electrode sensor is used. To read the data, the sensor must be charged to a voltage level adjustable in the microcontroller. The microcontroller, in turn, houses a comparator that generates a software interrupt when the level is exceeded. The readout essentially consists of reading the charge time of the formed capacitor from 0 to a certain level. In this embodiment of the EDA sensor, the strapping consists of 1 element on the board, making this circuit relatively simple to produce. However, if charging takes place with low current, the measurement signal can be significantly affected by noise induced currents, therefore in this case it is advisable to additionally provide the device with shielding means.

If the capacitance of the sensor described above is small (of order pF) and/or the capacitor formed in the device is charged with small currents, the sensor proposed above has a low reading resolution. The microcontroller has a timer unit that is large enough for a method based on charge rate measurement, therefore a resistor (strapping) is used to reduce the charge rate of the capacitor in this embodiment. In general, the larger the resistance of the resistor, the slower the charging of the capacitor is, and at the same time, the more noise will affect the measurement signal according to this embodiment.

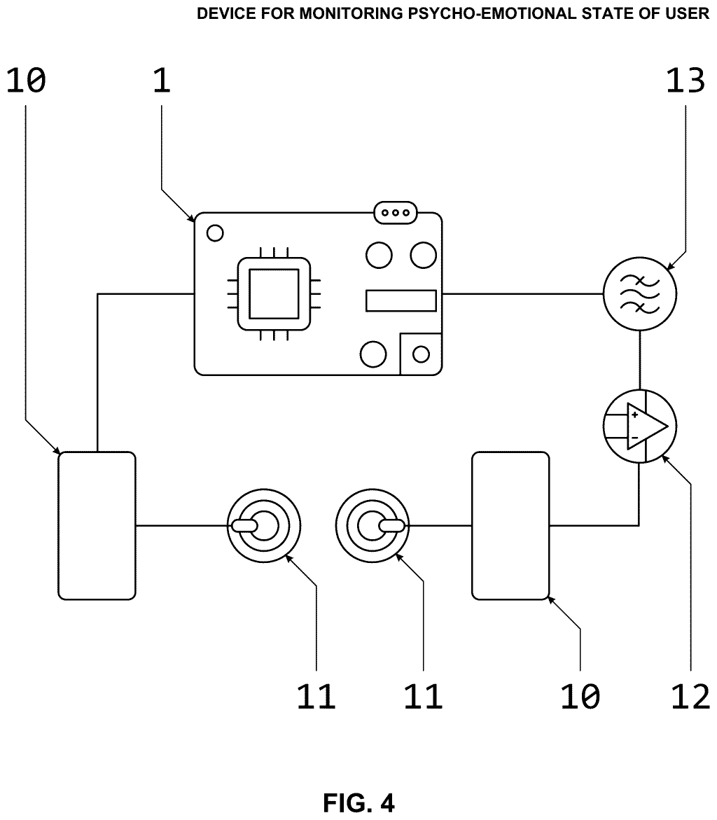

The second embodiment of the EDA sensor shown in FIG. 4 is based on reading the average discharge current of the capacitor. For this purpose, a current-limited rectangular signal is applied to the sensor by means of strapping, the capacitor is charged during the positive edge of the signal, and then the capacitor is discharged on the low edge. During discharge, the discharge current is averaged, amplified and transformed into voltage using an amplifier, after that the signal is filtered and fed to the ADC of the microcontroller.

There are also single-electrode non-contact sensors, contact sensors, including skin-galvanic sensors, and others that can read and transmit electrodermal activity information. For example, there are contact sensors that quantify sweating in the area where the sensor is located based on changes in the resistivity or conductivity of the surface layer of the skin. Non-contact sensors that measure changes in electrode potentials are also known. The present invention is not limited to the exemplary embodiments of electrodermal activity sensors and may include other variants thereof according to the art, the applicability of which in the present invention is obvious to those skilled in the art. However, it is necessary to consider the weight, size and power consumption of such EDA sensors in order to make the device wearable, and also that in the case of contact EDA sensors, contact between the wearable device and the user's body must be ensured.

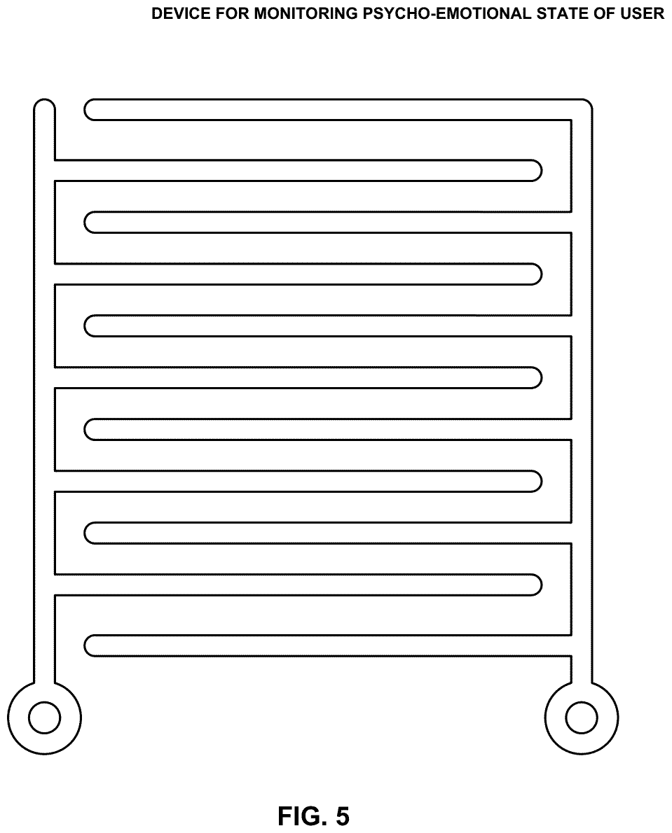

For achieving greater responsiveness of the sensor to changes in EDA parameters (small changes), the use of two electrodes is preferable. In this case, the electrodes of interdigital type are also preferable (one of possible variants of such type of electrodes is shown in FIG. 5), as such electrodes provide uniform intensity of electromagnetic field at some distance from the electrodes even in the presence of abundant sweating, got on the electrode, while use of other types of electrodes means a higher probability of uneven and strong penetration of electromagnetic field deep into the skin, which also contributes to the achieving of the technical result. Sweating affects the capacitor capacity by changing the electrical permeability of the skin.

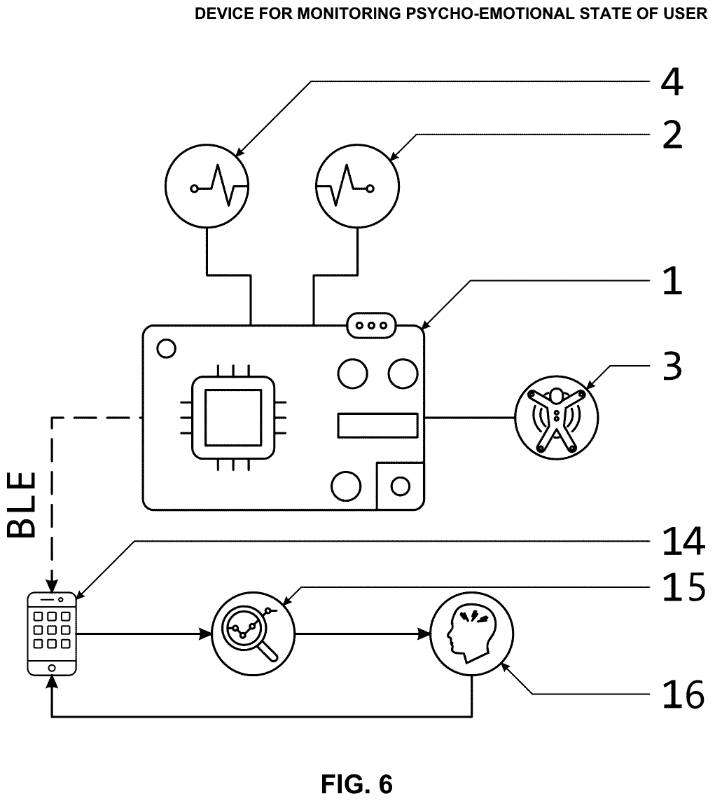

According to one embodiment, the device may be represented as a wristband fastened on the user's wrist and comprising an electronics unit (processor, charger, antenna and their strapping) and a sensor unit, an EDA sensor (examples were given above), a photoplethysmogram sensor and an accelerometer. Wireless data transmission is provided in the proposed wearable device, for example, in this embodiment it is implemented using the BLE (Bluetooth Low Energy) protocol (the functional diagram is shown in FIG. 6).

According to some embodiments, it is possible to transmit physiological parameters of the user, such as EDA, plethysmogram and accelerometer data, to a mobile application, after which the data are processed and the results are displayed to the user in real time via the mobile application. According to this embodiment, the complete cycle of operation of the proposed device includes the following steps: data from the sensors are received by the microcontroller, and then sent wirelessly to the mobile application; then the data from the mobile application are sent to the analysis unit (which unit, according to various embodiments, can be located either directly near the sensor housing or on a remote server); the analysis unit performs preprocessing and processing of the available data with the assessment of the psycho-emotional state of the user (according to some embodiments, this is, for example, a stress level on some universal scale (e. g., low-medium-high) that can be adjusted over the course of a single user's use of the device to match their stress levels); then the resulting assessment is sent to the user via the mobile application.

It should be noted that the term “mobile application” as used herein describes some means for displaying or indicating an assessment of a psycho-emotional state of a user, and may be in the form of a program on a mobile device, computer, or other device configured to receive and display data from the device for assessing a psycho-emotional state of a user, and is apparent to those skilled in the art.

The first step of data analysis according to one preferred embodiment is preprocessing, which plays an important role in analyzing and modeling biomarkers associated with psycho-emotional stress and includes a number of steps, such as:

Downsampling to ensure matching the time stamps of the data with different sampling rates;

Random sample reducing or sample discarding for unbalanced samples (where non-stressed samples dominate over stressed samples);

Standardization and normalization;

Cleansing data from outliers and anomalies.

The feature extraction process includes extracting relevant data from raw data that can be used as input features for analytical and machine learning models (Feature extraction). For example, in some embodiments, a decomposition into phase and tonic components is used for EDA data using a convex optimization approach with sliding windows ranging from 10 to 20 minutes. In yet another embodiment, the HRV analysis additionally uses HRV calculated from the P-P intervals of the photoplethysmogram to calculate temporal (such as SDNN, RMS-SD) and frequency (Total Power, Low/High Frequency) parameters. According to yet another embodiment, a vector of averaged accelerations within the observed time window, or a deviation of the acceleration vectors relative to each other is additionally calculated from the accelerometer data.

The Machine Learning model or analytical model takes the processed and selected features from the previous steps as input. The result of the work is a classification of psycho-emotional stress based on physiological parameters (Arousal Detection). An important part of solving the problem of determining and predicting a psycho-emotional state, as well as qualitative model training (in the embodiments where such a model is used), is the collection of relevant data, as well as their annotation. Data sets may be annotated by at least one of the following methods:

Through periodic annotation, when specific frames are labeled as stressful or non-stressful while the subject was in the appropriate state (e. g., relaxation, physiological stress, emotional stress, relaxation);

By having the user complete a self-assessment questionnaire;

By having the observer assess the level of stress and/or emotional state during the data collection period.

The listed methods can be used both in binary (presence/absence of stress) and multiclass classification tasks (e. g., emotion classification), and in regression tasks (e. g., determining the level of stress on some given scale).

Also, the general level of anxiety and tension at the time of the start of the experiment (data collection) is often important, and various techniques can be used for the assessment of this level, for example, such as the Spielberger-Hanin scale or the definition of the individual minute.

The following is an example of processing a data set from a device comprising an electrodermal activity sensor, a photoplethysmogram sensor, and an accelerometer, which data set was obtained over a 30 minute period with a following sequence of user's activities:

First, 6 minutes of slow walking (2 minutes of slow walking+2 minutes with the Stroop test+2 minutes of slow walking);

Next, 5 minutes in a relaxed state (rest);

Then 6 minutes of brisk walking (2 minutes brisk walking+2 minutes with the Stroop test+2 minutes brisk walking);

Then 5 minutes in a relaxed state (rest) again;

Next, 6 minutes standing (2 minutes standing+2 minutes with Stroop test+2 minutes standing);

And then 2 minutes in a relaxed state (rest).

According to one preferred embodiment of the invention, the first step in handling the data is preprocessing. In a basic embodiment of this step, it includes noise, outliers and anomalies removal.

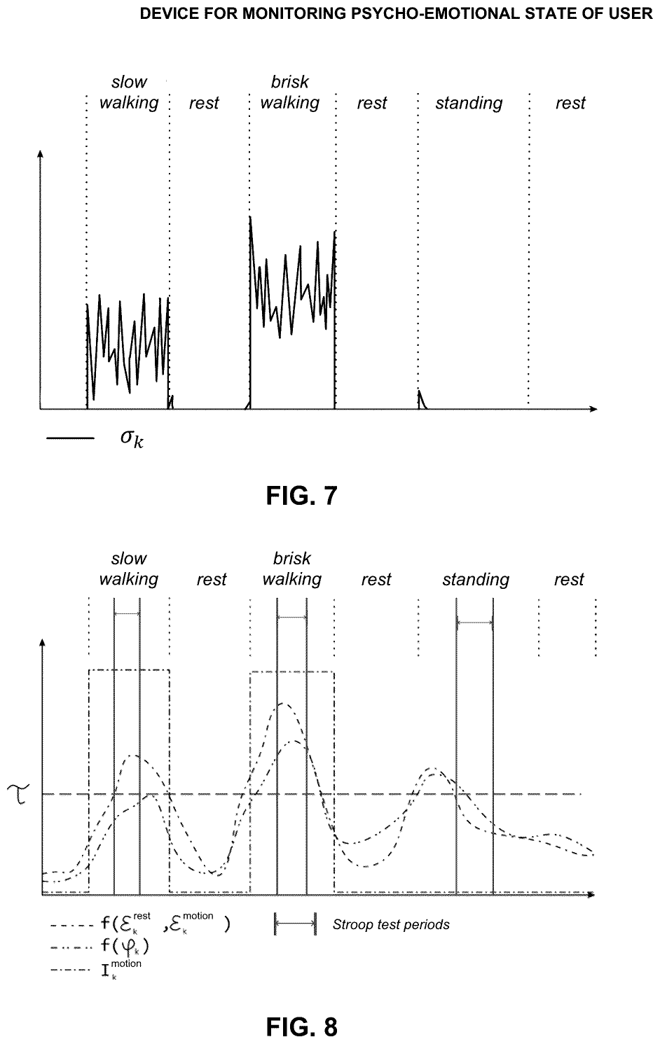

The next, second step of data processing by the analysis unit is Feature extraction. In the example under consideration, the acceleration parameters are calculated from the accelerometer data σₖ (see FIG. 7) in this step; the following parameters are calculated for each 5-minute window from the PPG data: calculation of P-P intervals, application of Kalman filter, and then parameters εₖ^{rest}, εₖ^{motion} are obtained (see FIG. 8); phase/frequency decomposition is applied to the electrodermal activity sensor data in a 5-minute window to obtain the φₖ graph (see FIG. 8).

Then, in the third step, which is the Movement Detection step, the Movement Indication parameter is calculated (see FIG. 8). Iₖ^{motion} (see FIG. 8) is based on the obtained acceleration parameters σₖ. If periods of physical activity are detected, it is necessary to perform the first step calculations again for the photoplethysmogram and electrodermal activity sensor data at time intervals corresponding to periods of activity, using stronger data filtering algorithms (this allows minimizing the error during the calculation of features in the second step), and then repeat the calculation of features in the second step and proceed to the third step.

After the performing above-mentioned processing according to the embodiment under consideration, a step of determining the level of emotional arousal (Arousal Detection) follows. A threshold value τ is selected, based on a classification according to a machine learning model or an analytical model, exceeding which by the graphs indicate emotional arousal, and the graph according to FIG. 8 is obtained as a result.

Bracketed reference items used in the present description and claims should not be construed as limiting the claims in any way. The word “comprising” does not exclude the presence of elements or steps other than those listed in the claim. An element in the singular does not exclude the presence of a plurality of such elements. The invention may be implemented by means of hardware comprising a plurality of individual elements and/or by means of a suitably programmed processor. In a claim enumerating multiple devices, some of these devices may be implemented by means of the same piece of hardware. The measures set forth in the different dependent claims may preferably be used in combination, it being understood by those skilled in the art that the principle underlying the achievement of the claimed technical advantages and the solution of the technical difficulties mentioned herein remains valid when combining the technical features of the claimed invention disclosed in the present description.

Therefore, the mentioned elements directly contribute to the technical result, which consists in expanding the capabilities of the device for monitoring a psycho-emotional state of a user.

The present application provides preferred embodiments of the claimed technical solution, which should not be construed as limiting other, particular embodiments thereof that do not go beyond the requested scope of legal protection and are obvious to those skilled in the art.

We claim:

1. A wearable device for monitoring data indicative of a psycho-emotional state of a user, the device comprises:
a photoplethysmogram sensor;
a microcontroller;
an accelerometer;
an electrodermal activity sensor;
an analysis unit configured to generate data for assessing the psycho-emotional state of the user on the basis of the signals of the said sensors;
wherein the analysis unit is configured to perform preliminary analysis of the data from the accelerometer and photoplethysmogram sensor to provide the determination of periods and levels of physical activity of the user during the said generation of data for assessing the psycho-emotional state of the user.

2. The wearable device for monitoring data indicative of a psycho-emotional state of a user according to claim 1, wherein the device additionally comprises at least one of the following sensors: a muscle activity sensor, a light sensor, a temperature sensor, a humidity sensor, a sound recording device, and wherein the analysis unit is configured to take into account the data received from the said sensors during the said preliminary analysis.

3. The wearable device for monitoring data indicative of a psycho-emotional state of a user according to claim 1, wherein the analysis unit is configured to be located separately from the wearable device, the analysis unit is configured to provide wireless communication with the wearable device.

4. The wearable device for monitoring data indicative of a psycho-emotional state of a user according to claim 1, wherein the analysis unit is configured to perform at least one of the following: downsampling to ensure matching the time stamps of the data, randomly reducing or discarding samples for unbalanced samples, cleansing the data from outliers and anomalies.

5. The wearable device for monitoring data indicative of a psycho-emotional state of a user according to claim 1, wherein the analysis unit is configured to perform feature extraction including at least the following:
decomposition into phase and tonic components using a convex optimization approach with sliding windows ranging from 10 to 20 minutes for electrodermal activity sensor data;
calculation of heart rate variability by P-P intervals of photoplethysmogram and its use for calculation of parameters of temporal and frequency analysis of heart rate variability;
calculation of the vector of averaged accelerations within the observed time window or deviation of acceleration vectors from each other.

6. The wearable device for monitoring data indicative of a psycho-emotional state of a user according to claim 1, wherein the analysis unit includes a model trained on relevant annotated data.

7. The wearable device for monitoring data indicative of a psycho-emotional state of a user according to claim 1, wherein the electrodermal activity sensor is a two-electrode sensor with the electrodes of the interdigital type.

8. The wearable device for monitoring data indicative of a psycho-emotional state of a user according to claim 1, wherein the said preliminary analysis of the data in the analysis unit is performed using downsampling the signals received.

9. The wearable device for monitoring data indicative of a psycho-emotional state of a user according to claim 1, wherein the device comprises a data transmission module.

10. A method of analyzing, processing and correcting data received from the device for monitoring a psycho-emotional state of a user according to claim 1, wherein:
data preprocessing is performed using the analysis unit;
relevant data are extracted from the processed data using the analysis unit;
periods of user's movements are detected based on the relevant data using the analysis unit;
periods of user's movements are subjected to re-processing with stronger data filtering algorithms using the analysis unit;
relevant data are re-extracted from the processed data within the periods of user's movements using the analysis unit;
the extracted data are fed to a machine learning or analytical model using the analysis unit;
psycho-emotional state classifying is performed based on the extracted data using the machine learning or analytical model;
graph of the user's psycho-emotional state is plotted.

11. The method for analyzing, processing and correcting data received from the device for monitoring a psycho-emotional state of a user, according to claim 10, wherein at least one of the following is performed in the step of data preprocessing:
downsampling to ensure matching the time stamps of the data;
randomly reducing or discarding samples for unbalanced samples;
cleansing the data from outliers and anomalies.

12. The method for analyzing, processing and correcting data received from the device for monitoring a psycho-emotional state of a user, according to claim 10, wherein the feature extraction is performed in the step of relevant data extracting, the feature extraction includes at least the following:
decomposition into phase and tonic components using a convex optimization approach with sliding windows ranging from 10 to 20 minutes for electrodermal activity sensor data;
calculation of heart rate variability by P-P intervals of photoplethysmogram and its use for calculation of parameters of temporal and frequency analysis of heart rate variability;
calculation of the vector of averaged accelerations within the observed time window or deviation of acceleration vectors from each other.

13. The method for analyzing, processing and correcting data received from the device for monitoring a psycho-emotional state of a user, according to claim 10, wherein Hampel filter method is used for preprocessing with stronger data filtering algorithms.

14. The method for analyzing, processing and correcting data received from the device for monitoring a psycho-emotional state of a user, according to claim 10, wherein the graph of the psycho-emotional state of the user is additionally transmitted to a mobile application by means of a data transmission module.

15. The method for analyzing, processing and correcting data received from the device for monitoring a psycho-emotional state of a user, according to claim 10, wherein relevant data are collected and annotated to classify the psycho-emotional state of the user.

16. The method for analyzing, processing and correcting data received from the device for monitoring a psycho-emotional state of a user, according to claim 15, wherein at least one of the methods is used to annotate relevant data:
periodic annotation;
filling out a self-assessment questionnaire by the user;
the observer's assessment of the level of stress and/or emotional state during the data collection period.

17. The method for analyzing, processing and correcting data received from the device for monitoring a psycho-emotional state of a user, according to claim 15, wherein the general level of anxiety and tension at the time of the start of the experiment (data collection) is additionally determined by at least one of the methods:
method using the Spielberger-Hanin scale;
method with determining the individual minute.
